(19) United States
(12) Patent Application Publication
(10) Pub. No.: US 2013/0178704 A1
(43) Pub. Date: Jul. 11, 2013

(54) Scope Tubes, Intubation Assemblies, and Methods of Intubation

(71) Applicant: Francisco JAIME, Albuquerque, NM (US)

(72) Inventor: Francisco JAIME, Albuquerque, NM (US)

(21) Appl. No.: 13/393,830

(22) Filed: Mar. 1, 2012

Related U.S. Application Data
(63) Continuation of application No. PCT/US10/47593, filed on Sep. 1, 2010.
(60) Provisional application No. 61/239,058, filed on Sep. 1, 2009.

(51) Int. Cl. A61M 16/04; A61B 1/267; A61B 1/00

(52) U.S. Cl. CPC A61M 16/0488; A61B 1/00165; A61B 1/00066; A61B 1/267
USPC 600/120; 600/182

(57) ABSTRACT

A scope tube may include an elongated member, a lever, a coupling member, and a handle portion. The elongated member may define a through bore tapering from a proximal end of the elongated member toward a distal end of the elongated member. The lever may be pivotally coupled to the elongated member, and the coupling member may be pivotally coupled with the lever and may extend beyond the proximal end of the elongated member. The handle portion may be fixedly coupled with the elongated tube distal of the lever. The handle portion may be proximate the lever such that a user can grasp the handle portion with one hand and operate the lever with the thumb of the same hand.

### CROSS-REFERENCE TO RELATED APPLICATIONS

This application claims the benefit of priority of U.S. Provisional Patent Application No. 61/239,058, filed on Sep. 1, 2009, the disclosure of which is incorporated herein by reference.

### TECHNICAL FIELD

The present disclosure relates to scope tubes and, more particularly, to a scope tube slidable over a fiberscope and configured to facilitate manipulation of the fiberscope and articulation of the distal tip of the fiberscope.

### BACKGROUND

One of the most important roles of the anesthesiologist is maintaining the patient's airway open and patent during the delivery of general anesthesia. An endotracheal tube (ETT) is placed into the mouth, through the vocal cords, and into the trachea in order to maintain a patent airway and effectively oxygenate and ventilate the patient. To accomplish this, a metal laryngoscope is generally placed into the patients' mouth and used to lift the tongue and expose the vocal cords. This is referred to as direct laryngoscopy. The ETT is then passed through the vocal cords and into the trachea in a procedure known as “intubation” of the trachea. The ventilation circuit is then attached to the patient to allow oxygenation and ventilation of the patients' lungs.

Some patients are easy to intubate while others are more difficult. Occasionally the vocal cords cannot be visualized even with direct laryngoscopy by an experienced anesthesiologist. This situation would be even more common with less experienced health care providers including physicians, paramedics, medics, etc who may need to intubate a patient outside of the operating room, in a clinic, at an accident scene, or even on the battlefield. An instrument that can see around the tongue and indirectly visualize the vocal cords would allow for intubation of the trachea in this situation.

A fiberoptic scope can be used in this case. This is a long snake like instrument that has a light and camera on the tip and a tip that is maneuverable. Unfortunately the fiberscope requires the use of both hands to properly manipulate. One hand is used to operate a lever or other mechanism, typically located at the proximal end of the fiberscope, to maneuver the tip, while the second hand is positioned more distally and used to position the fiberscope. In a difficult airway situation, the left hand would have to release the laryngoscope in order to properly manipulate the fiberscope with two hands. Unfortunately this would allow the tongue to fall back done into the airway and obstruct visualization.

It may be desirable to provide a mechanism by which the fiberscope could be manipulated with one hand alone while the other hand can continue to hold the laryngoscope and maintain the tongue elevated and out of the way and providing maximal possible exposure.

### SUMMARY

According to various aspects of the disclosure, a scope tube may include an elongated member, a lever, a coupling member, and a handle portion. The elongated member may define a through bore tapering from a proximal end of the elongated member toward a distal end of the elongated member. The lever may be pivotally coupled to the elongated member, and the coupling member may be pivotally coupled with the lever and may extend beyond the proximal end of the elongated member. The handle portion may be fixedly coupled with the elongated tube distal of the lever. The handle portion may be proximate the lever such that a user can grasp the handle portion with one hand and operate the lever with the thumb of the same hand.

In some aspects, an intubation assembly may include a scope tube, a fiberscope slidably received by and extending through the scope tube, and an endotrachial tube removably coupled with the scope tube and mounted over the fiberscope. The fiberscope may include a lever coupled to a distal tip of the fiberscope such that movement of the lever manipulates the distal tip of the fiberscope. The scope tube may include a lever coupled to the fiberscope lever such that movement of the scope tube lever causes movement of the fiberscope lever to manipulate the distal tip of the fiberscope.

According to some aspects, a method of intubating a trachea may comprise sliding a fiberscope into and through a scope tube, removably coupling an endotrachial tube with the scope tube and over the fiberscope, maneuvering the scope tube to position the fiberscope and endotrachial tube proximate a patient's trachea; and sliding the endotrachial tube off the scope tube and into the trachea. In some aspects, the method may include moving a lever associated with the scope tube to manipulate a distal tip of the fiberscope.

### BRIEF DESCRIPTION OF THE DRAWINGS

FIG. 1 is a schematic illustration of an exemplary scope tube in accordance with various aspects of the disclosure.

FIG. 2 is a schematic illustration of an exemplary intubation assembly in accordance with various aspects of the disclosure including the scope tube of FIG. 1.

FIG. 3 is a schematic illustration of a distal end of the exemplary intubation assembly of FIG. 2.

FIG. 4 is a schematic illustration of the exemplary intubation assembly of FIG. 2 at use in a patient.

### DETAILED DESCRIPTION OF VARIOUS EMBODIMENTS

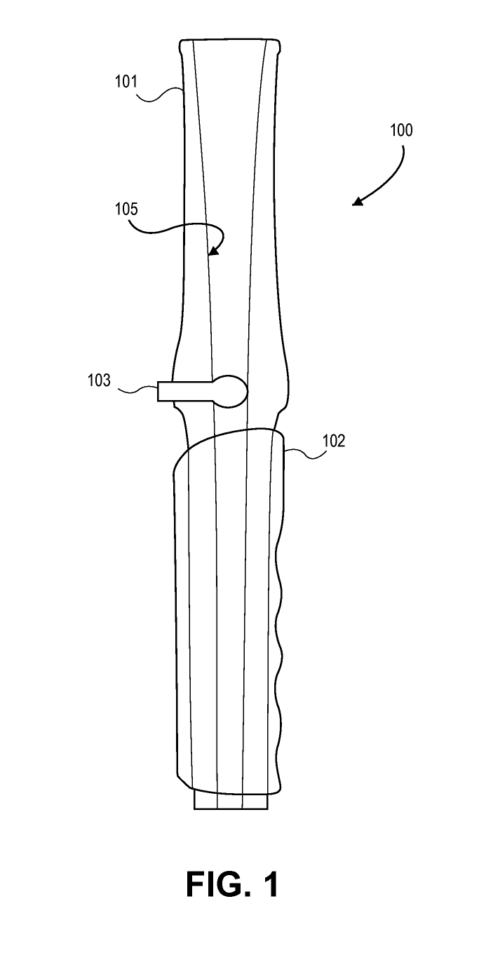

FIG. 1 illustrates an exemplary scope tube 100 in accordance with various aspects of the disclosure. The scope tube 100 includes a tube portion 101, a handle portion 102, and a lever 103. The tube portion 101 extends through the handle portion 102 and includes a through bore 105 for receiving a fiberscope. The handle portion 102 is fixedly coupled to the tube portion 101. The lever 103 is pivotally coupled with the tube portion 101. The scope tube 100 may be constructed from any material approved for medical use, as described herein. For example, the scope tube 100 may be constructed of metal, plastic, or a combination thereof. In some aspects, the tube portion 101, the handle portion 102, and the lever 103 may be constructed of a plastic material, while the means for coupling these elements together may be constructed of a metal material. For example, the lever 103 may be pivotally coupled with the tube portion 101 via a metal pin. Other material constructions of the elements of the scope tube 100 would be understood by persons skilled in the art and are contemplated as part of the present disclosure.

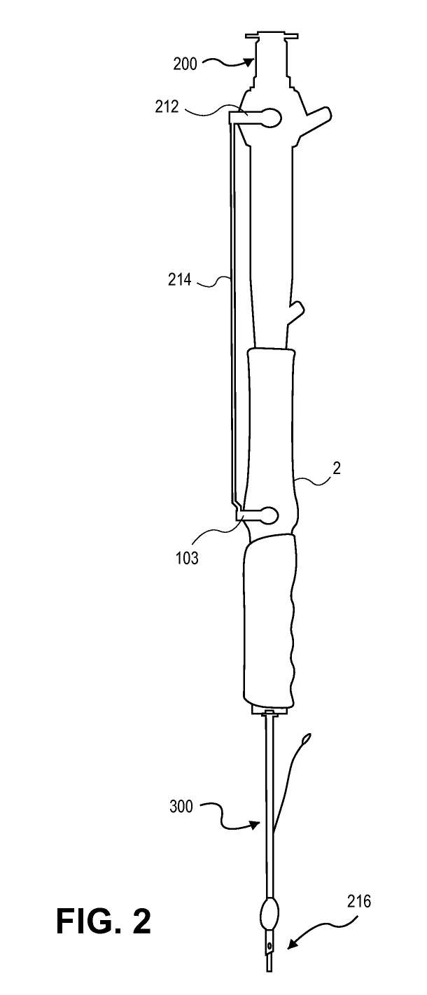

Referring now to FIG. 2, a fiberscope 200 may be inserted into the scope tube 100. For example, the throughbore 105 of the scope tube 100 may be structured and arranged to slidably receive any conventional fiberscope 200. In some aspects, the fiberscope 200 may be coupled to the scope tube 100 via a snug fit or friction fit relationship at a proximal region of the scope tube 100 (i.e., at the end of the scope tube into which the fiberscope is inserted). In some aspects, the scope tube 100 and/or the fiberscope 200 may include a coupling assembly for securely coupling the fiberscope 200 with the scope tube 100 once the fiberscope is inserted therein. In any event, the scope tube 100 and the fiberscope 200 shall be coupled together in a stable manner such that the scope tube 100 allows the fiberscope 200 to be easily held and maneuvered with one hand, for example, by gripping the handle portion 102 of the scope tube 100.

An endotrachial tube 300 may also be coupled to the scope tube 100 in a manner such that movement of the scope tube 100 will cause like movement of the endotrachial tube 300. For example, the fiberscope 200 may be inserted into the scope tube 100. The fiberscope 200 may then extends out a distance from the distal end of the scope tube 100. The endotrachial tube 300 may then be railroaded over the fiberscope and secured in place by the lumen of an endotrachial tube connector slipping over another short (1 cm at most) segment of tube that is at the distal end of the scope tube 100. According to some aspects, the fiberscope 200 may extend out about an inch from the distal end of the endotrachial tube 300.

The fiberscope 200 may comprise a conventional fiberscope including a lever 212 operable by a user to articulate a distal tip 216 of the fiberscope, as would be understood by persons skilled in the art. The lever 103 of the scope tube 100 may be coupled to the lever 212 of the fiberscope 200 via a coupling member 214, for example, a thin rod or the like, which extends proximally beyond a proximal end of the scope tube 100. According to various aspects, the coupling member 214 may have a variable length, which may be adjustable so as to accommodate conventional fiberscopes 200 of various different sizes and configurations. For example, the coupling member 214 may comprise two or more telescoping tubes, as would be understood by persons skilled in the art. In some aspects, the telescoping tubes may be locked relative to one another once coupled with the levers 103, 212, as would be understood by persons skilled in the art.

In some aspects, the coupling member 214 may be integrally formed with the lever 103 of the scope tube 100 as a single-piece of unitary construction. In such aspects, a proximal end of the coupling member 214 may be coupled to the lever 212 of the fiberscope 200 by any conventional means. For example, the proximal end of the coupling member 214 may include a housing for receiving the lever 212 in a snug fit or friction fit relationship. In some aspects, the coupling member 214 may be coupled to the lever 212 via a snap fit assembly, a set screw assembly, or any other conventional coupling assembly. According to various aspects, the coupling member 214 may be constructed separate from the levers 103, 212. In such aspects, a proximal end of the coupling member 214 may be coupled to the lever 212 of the fiberscope 200 by any conventional coupling assembly, as described above, and a distal end of the coupling member 214 may be coupled to the lever 212 of the fiberscope 200 by any conventional coupling assembly, such as those described above in connection with the proximal end.

As would be understood by persons skilled in the art, the coupling member 214 is pivotally coupled with the levers 103, 212 such that the distance between the levers 103, 212 remains constant as the lever 103 is pivoted relative to the scope tube 100 and the lever 212 is simultaneously pivoted relative to the fiberscope 200. For example, the coupling member 214 may include a distal end portion pivotal relative to the elongated rod portion, as well as a proximal end portion pivotal relative to the elongated rod portion. The elongated rod portion extends in a direction substantially parallel to a longitudinal dimension of the scope tube 100. Then, the distal end portion of the coupling member 214 is coupled to the lever 103 and the proximal end portion of the coupling member is coupled to the lever 212, such that the elongated rod portion of the coupling member 214 is pivotal relative to the lever 103 and the lever 212. As a result, pivoting motion of either lever 103, 212 causes translational movement of the coupling member 214 substantially along an axis parallel with a longitudinal dimension of the scope tube 100. Also, movement of either lever 103, 212 causes like movement of the other lever.

In operation, once the lever 103 of the scope tube 100 is coupled with the lever 212 of the fiberscope 200 via the coupling member 214, movement of the scope tube lever 103 causes a similar movement of the fiberscope lever 212, while the endotrachial tube 300 is just passively along for the ride. It should be appreciated that the fiberscope lever 212 may be coupled to a distal tip 216 of the fiberscope 200 by any known mechanism such that movement of the fiberscope lever 212 causes articulation/flexion of the distal tip 216 of the fiberscope 200. Thus, movement of the scope tube lever 103 may cause articulation/flexion of the distal tip 216 via the coupling member 214 and the fiberscope lever 212.

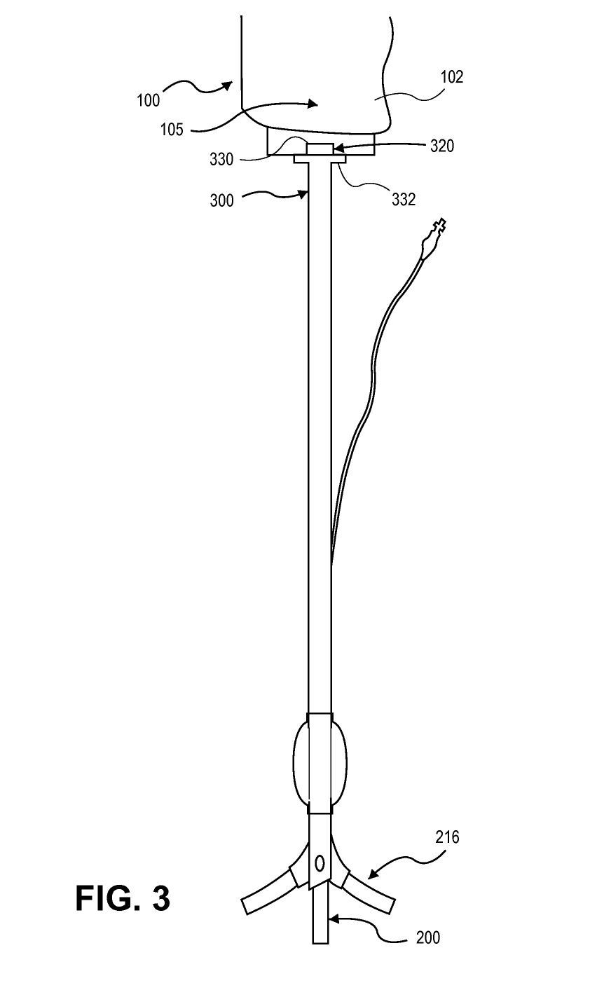

Referring now to FIG. 3, the scope tube 100 may include a bore 320 at its distal end. The bore 320 may have a larger cross-section than that of the throughbore 105. The endotrachial tube 300 may have a proximal end portion 330 sized to snugly fit (i.e., a friction fit) into the bore 320 such that movement of the scope tube 100 causes like movement of the endotrachial tube 300. It should be appreciated that the endotrachial tube 300 may be coupled to the scope tube 100 via any conventional coupling assembly including, but not limited to, a screw fit, a key fit, a set screw, or the like. The endotrachial tube 300 may also include a flange portion 332 that may rest against the distal end of the scope tube 100 when the proximal end portion 330 is received by the bore 320. In some aspects, the endotrachial tube 300 may include a lever or other known ejection member configured and arranged relative to the scope tube 100 so as to facilitate removal of the endotrachial tube 300 from the scope tube 100. In some aspects, the scope tube 100 may include a lever or other known ejection member configured and arranged relative to the endotrachial tube 300 so as to facilitate removal of the endotrachial tube 300 from the scope tube 100. In any event, the endotrachial tube 300 must be capable of being slid off of the scope tube 100 when in an appropriate position to be passed into the trachea.

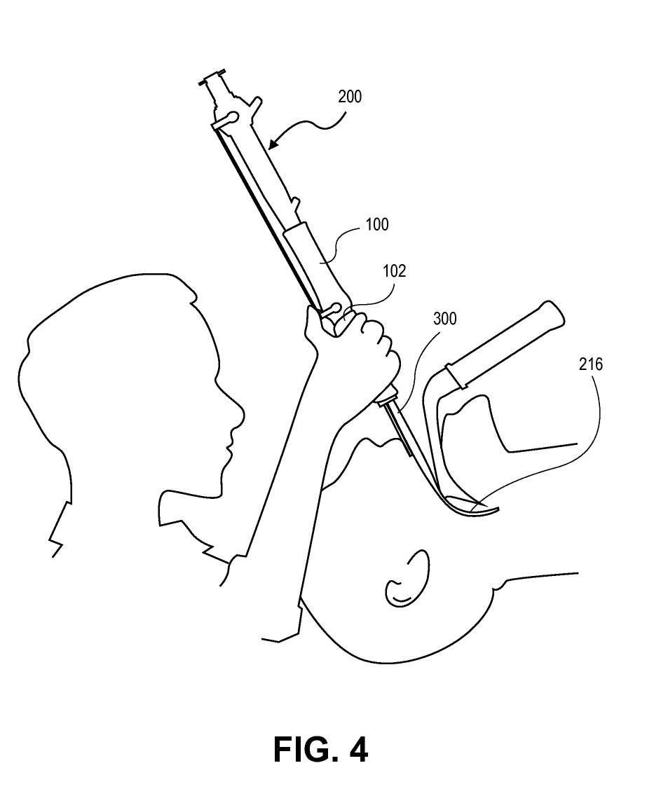

In use, as shown in FIG. 4, one hand of a user can be used to hold the laryngoscope and lift the tongue up. If the vocal cords are not directly visualized via plain sight, the user's other hand can then hold the fiberscope 200 with the scope tube 100 via handle portion 102 and the fiberscope 200 can be easily manipulated around the base of the tongue. The vocal cords can then be indirectly visualized (e.g., via a monitor operationally associated with the fiberscope) and the fiberscope tip 216 and endotrachial tube 300 can be passed through the vocal cords and into the trachea. The endotrachial tube 300 can then be slid off the fiberscope 200 and into the trachea. The trachea is therefore successfully intubated even though the vocal cords were not visualized directly with direct laryngoscopy.

Thus, the scope tube may simplify tracheal intubation done by inexperienced health care providers. The scope tube may also simplify intubation of difficult airways done by experienced and inexperienced health care providers including anesthesiologists. The scope tube may allow limited movement of the neck in patients with known or suspected cervical spine injuries, thereby serving to avoid injury to a patient's spinal cord in these situations.

It will be apparent to those skilled in the art that various modifications and variations can be made to the scope tubes, intubation assemblies, and methods of intubation of the present disclosure without departing from the scope of the invention. Other embodiments of the invention will be apparent to those skilled in the art from consideration of the specification and practice of the invention disclosed herein. It is intended that the specification and examples be considered as exemplary only.

What is claimed is:

1. A scope tube, comprising:
an elongated member defining a through bore tapering from a proximal end of the elongated member toward a distal end of the elongated member;
a lever pivotally coupled to the elongated member;
a coupling member pivotally coupled with the lever and extending beyond the proximal end of the elongated member;
a handle portion fixedly coupled with the elongated tube distal of the lever, the handle portion being proximate the lever such that a user can grasp the handle portion with one hand and operate the lever with the thumb of the same hand.

2. The scope tube of claim 1, wherein the coupling member is structured and arranged to couple the lever with a second lever associated with a fiberscope such that movement of the lever causes like movement of the second lever.

3. An intubation assembly, comprising:
a scope tube in accordance with any of the preceding claims;
a fiberscope slidably received by and extending through the scope tube; and
an endotrachial tube removably coupled with the scope tube and mounted over the fiberscope.

4. The assembly of claim 3, wherein the fiberscope includes a second lever coupled to a distal tip of the fiberscope such that movement of the lever manipulates the distal tip of the fiberscope.

5. The assembly of claim 4, wherein movement of the scope tube lever causes movement of the fiberscope lever to manipulate the distal tip of the fiberscope.

6. A method of intubating a trachea, comprising:
sliding a fiberscope into and through a scope tube;
removably coupling an endotrachial tube with the scope tube and over the fiberscope;
maneuvering the scope tube to position the fiberscope and endotrachial tube proximate a patient's trachea; and
sliding the endotrachial tube off the scope tube and into the trachea.

7. The method of claim 6, further comprising moving a lever pivotally coupled with the scope tube to manipulate a lever pivotally coupled with the fiberscope in order to articulate a distal tip of the fiberscope.
